(19) United States
(12) Patent Application Publication
(10) Pub. No.: US 2002/0025349 A1
(43) Pub. Date: Feb. 28, 2002

(54) Novel herbal composition for diabetes patients and a process for producing the same

(76) Inventors: Narasimha Baba Brindavanam, Ghaziabad (IN); Chandrakant Katiyar, Ghaziabad (IN); Yadlapalli Venkateswara Rao, Ghaziabad (IN)

Correspondence Address: Bernhard D. Saxe, FOLEY & LARDNER, Washington Harbour, 3000 K Street, N.W., Suite 500, Washington, DC, 20007-5109, US

(21) Appl. No.: 09/845,723

(22) Filed: May 2, 2001

(30) Foreign Application Priority Data
May 2, 2000 (IN) 478/DEL/2000

(51) Int. Cl. A61K 35/78

(52) U.S. Cl. 424/757; 424/758; 424/762

(57) ABSTRACT

The invention provides a synergistic oral liquid herbal composition falling under the category of “Asavas” and “Arishtas”, useful for management of diabetes, said composition comprising a therapeutically effective amount of plant extracts, self-generated ethanol to the extent of 7 to 12% v/v and having not more than 1 to 3% w/w of sugar content and also provides a novel method for the manufacture of herbal compositions in liquid oral dosage form containing a limited amount of self generated alcohol.

### FIELD OF THE INVENTION

The invention provides a synergistic oral liquid herbal composition falling under the category of “Asavas” and “Arishtas”, useful for management of diabetes, said composition comprising a therapeutically effective amount of plant extracts, self-generated ethanol to the extent of 7 to 12% v/v and having not more than 1 to 3% w/w of sugar content. This invention also provides a novel method for the manufacture of herbal compositions in liquid oral dosage form containing a limited amount of self generated ethanol. This process facilitates the production of fermented liquid orals virtually free from sugar and hence provides benefits to the large segment of population suffering from Diabetes. The invention also relates to the unique herbal composition for reducing the blood sugar levels in the mammals especially humans suffering from diabetes.

### BACKGROUND OF THE INVENTION

Diabetes

Diabetes Mellitus results from diminished secretion of insulin by the beta cells of the islets of langerhans. Factors responsible for causing Diabetes are Heredity and Obesity. Heredity increases the susceptibility of beta cells to viral invasions or favor the development of autoimmune antibodies against the beta cells, thus leading to their destruction. Obesity decreases the number of insulin receptors in the insulin target cells throughout the body. Hence, the amount of insulin present is inadequate to induce its usual metabolic effects.

In diabetic patients blood glucose levels goes as high as 1200 mg/dl are known to occur which is 12 times higher than the normal. Levels of 300 mg/dl to 500 mg/dl are common in diabetic patients. Thus the tendency of diabetes is to cause both extra-cellular and intracellular dehydration to develop (Guyton, Ca & Hall Ej (1996): In Text Book Of Medical Physiology, 9^{th} Ed., Chapter 78, “Insulin, Glucagon And Diabetes Mellitus”, (Prism Indian Edition), Prism-Saunders, Bangalore, Pp 980 -983).

Diabetic Symptoms, which may arise due to pathological physiology of Insulin lack, are Polyuria: due to the osmotic diuretic effect of glucose in the kidney tubules; Polydipsia:due to dehydration resulting from polyuria; loss of weight: The failure of Glucose and protein metabolism by the body; Polyphagia: Loss of weight causes tendency of eating more; Asthenia: Loss of body protein and diminished utilization of carbohydrates for energy.

The usual methods for diagnosing diabetes are based on various chemical tests of urine and the blood viz, Urinary Sugar, Fasting Blood Glucose Level, Glucose Tolerance Test and Acetone breath.

Complications of disease:Diabetes Mellitus involves many organs systems of the human body leading to many systemic complications like Diabetic Neuropathy, Diabetic Diarrhoea, Urinary retention, Gustatory Swelling, Pupillary Reflexes, Cardiac Autonomic Disturbances, Collagen Disturbances. Of these disorders Diabitic Neuropathy is the most common and affects patients at earlier stages. Also this particular complication has a major relevance to the context.

Diabetic Neuropathies are of following types (Bell Ji & Hockaday Tdr (1996): In Oxford Textbook Of Medicine, Ed. By Weatherall, Dj, Ledingham Jgg & Warell Da, Chapter 11.11 “Diabetes Mellitus”, 3^{rd} Ed., Oxford University Press, Pp-1487-1490.):

1. Radiculopathy involving the Nerve Root.

2. Mono Neuropathy which involves Mixed Spinal or Cranial and NerveTerminal and affects Single Dermatone, Ann, Leg, Cranial nerves III, IV, VI, X, XII

3. Poly Neuropathy involves Sensory and Motor Fibres. The part affected is Feet.

4. Amvotrophy involves Motor Fibres and the main parts affected are Quadriceps, Gluteal muscles, Hamstrings

5. Autonomic Neuropathy involves Sympathetic Ganglia and Fibres affecting Cardiovascular, Gastrointestinal, Bladder and causing Impotence.

Herbal Drugs for Diabetes

In Ayurveda several herbal ingredients are mentioned for the treatment of diabetes (Madhumeha). Herbal ingredients such Gurmar leaves (Gymnema sylvestre), Methi seeds (Trigonella foenumgrecum), Vijayasar heartwood (Pterocarpus marsupium), Jamun seeds (Eugenia jambolana), Karela (Momordica charantia) etc are few examples in this category. Various scientific investigations on these plants suggest their role in the care of diabetics (Atta-Ur-Rahman & Khurshidzaman, Journal Of Ethnopharmacology, 26 (1989); 1-55.). Many of the herbal formulations for diabetes available in the market are perhaps based on these leads.

The hypoglycemic properties of Momordica charantia has been widely reported. Raza et al has reported the effect of oral feeding of Karela fruit juice on the Hepatic Cytochrome P 450 (CYP) and Glutathione S-transferase (GST) drug metabolising enzymes in the Streptozotocin induced diabetic rats (Raza H; Ahmed I; Lakhani Ms; Sharma Ak; Pallot D & Montague W, Biochem Pharmacol, 1996, Nov 22, 52 (10): 1639-42). Day et al has reported that the aqueous extract of Momordica charantia lowered the glycemic response to both oral and intraperitoneal glucose without affecting the insulin response (Day C; Cartwright T; Provost J & Bailey Cj,; Planta Med;1990 October;56(5): 426-9.). Yet another study reported that the water soluble extract of the Karela fruits increases the glucose tolerance (Leatherdale Ba; Panesar Rk; Singh G; Atkins Tw; Bailet Cj & Bingnell Ah; Br.Med J(Clin Res Ed.) 1981 June 6; 282 (6279): 1823-4).

Eugenia jambolana commonly known as Jamun is widely used in Indian folk medicine for the treatment of diabetes mellitus (Prince Ps; Menon Vp & Pari L; J Ethnopharmacol ;1998 May; 61 (1): 1-7). It has been reported that the aqueous extract of Jamun seeds has hypoglycemic action.

Several authors reported anti-diabetic principles of Gymenma sylvestre. Persaud et. al has reported that the alcoholic extract of G. Sylvestre stimulates the insulin release (Persaud Sj; Al-Majed H; Raman A & Jones Pm; J Endocrinol 1999 November; 163(2): 207-12). In another study Baskaran et al has reported the role of Gymenma sylvestre extract in type 2 diabetic patients (Baskaran K; Kizar Ahnmath B; Radha Sk & Shanmugasundarm Er; J Ethnopharmacol 1990 October; 30(3): 295-300). Yet another study conducted on 27 patients revealed the role of water soluble extract of G.sylvestre on insulin dependent diabetes mellitus (IDDM) (Shanmugasundarm Er; Rajeswari G Et Al; J Ethnopharmacol 1990 October; 30(3): 281-94).

Numerous reports are available on the hypoglycemic activity of Trigonella foenumgrecum (Methi). In a randomized, crossover metabolic study, Sharma et al reported that a significant decrease in the blood glucose levels in non-insulin dependent diabetic patients (Sharma Rd & Raguram Tc; Nutr Res 1990; 10:731-739). Yet another study reported that a decoction and an ethanolic extracts of Methi seeds were significantly decrease blood glucose in normal and alloxan-diabetic mice (Ajabnoor Ma & Tilmisany Ak; J Ethanopharmacol 1998; 22:45-49).

Anti-diabetic principles of Pterocorpus marsupium have been reported and the active principles of Vijaysar also identified. Indian Council of Medical Research (ICMR) had conducted a clinical trials on Vijayasar (Pterocarpus marsupium) in the treatment of non-insulin dependent diabetes mellitus (NIDDM) (ICMR; Indian J Med Res 1998 July; 108:24-9). Yet another study reported the hypoglycemic activity of an active constituents of Pterocarpus morsupium (Sheehan Ew, Zemaitis Ma, Slatkin Dj & Schiff P1 Jr; J Nat Prod 1983 March-April; 46(2): 232-4).

It is noteworthy that the various herbal antidiabetic formulations available in market place comprise of one or more of the drugs reviewed above. Almost all of these formulations are presented as solid oral dosage forms like Capsules, Tablets, Powders only.

Liquid Orals in Ayurveda

Asavas and Arishtas comprise an important group of liquid orals mentioned in ancient Ayurvedic literature. This dosage form offers the advantages of accessibility, palatability and product stability over the traditionally used decoctions, which are primitive in their nature. This particular group of liquid orals finds their use not only in Ayurveda, but also in other traditional systems of medicine practiced in neighboring oriental countries like Sri Lanka, Tibet, China and Bhutan. A fairly large number of Ayurvedic formulations described in ancient and mediaeval Ayurvedic literature fall under this group. Various Ayurvedic pharmaceutical companies in India do manufacture almost 50 different formulations falling under this range and some of these formulations are also being exported to other countries. To an estimate, every year Rs. 800 millions worth of Asava & Arishta group of formulations are being consumed in India alone.

Process of Asavas/Arishtas: An outline

Asava—Arishta group of formulations is being manufactured as per the procedures laid down in ancient texts. This procedure involves mainly three phases: 1) Extraction 2) Preparation of fermentation medium 3) Inoculation and fermentation and these three phases are summarized as below:

Extraction Phase: Water is used as solvent for extraction purposes. For Asavas, the extraction procedure involves a cold infusion technique. Therefore, the coarse powder of herbal material is soaked in water for a fixed duration at room temperature and a filtrate is obtained. In case of Asishtas, the extraction procedure involves an Open Pan Boiling Technique. Accordingly, the coarsely ground herbal material is charged to a boiling pan along with required quantity of water. This mixture is then, heated using steam or firewood or some other fuel. The filtrate is then collected discarding the herbal marc.

Preparation of fermentation medium: To grow any microorganism nutrients are necessary. As the extract mainly contains active constituents coming from herbs, Jaggery was suggested as a source of complex nutrition in ancient literature. Accordingly, the Fermentation medium is to be prepared by dissolving Jaggery in the herbal extract obtained either by cold infusion for Asavas or by means of Open Pan Boiling Technique for Arishtas.

Inoculation & Fermentation: Ancient literature recommends two herbs-viz. Woodfordia fruticosa and Madhuca latifolia as the inoculum bearing herbs. One or both of these two inoculum-bearing herbs and other aromatic herbs in form of fine powders are topped to the fermentation medium. Fermentation is carried out in a closed fermentation vessel, under controlled temperature conditions, at 30-35° C. for a period of 40-45 days. The end product is expected to contain a self-generated alcohol in range of 7 to 12% v/v.

The herbal product range produced as per the above process are termed as Asava or Arishta as the case may be. The product line has certain specific advantages from clinical application and stability points of view and the same can be summarized as under:

Products prepared under the process are generally palatable for the user with a sweet taste combined with a fine spicy aroma, which masks the unacceptable taste and odour of the active, herbal ingredients.

Apart from the aqueous extract of the prime ingredients, the preparation brings the hydro-alcoholic extract of supportive ingredients used as powders added during fermentation.

The process also brings, the extract of inoculum bearing herbs, which is considered to potentiate the activity of prime ingredients.

The self-generated alcohol content in the product acts as a preservative and contributes for its prolonged shelf life.

Self-generated alcohol is considered to enhance the bioavailability of all the herbal ingredients contained in the formulation.

As seen from the above information Asavas and Arishtas have many advantages and they constitute a brilliant pharmaceutical concept. While Asavas and Arishta formulations are available for wide range of therapeutic applications, diabetes apparently is an exception. Ayurvedic literature sparingly offers an herbal anti-diabetic formulation in this conventional dosage form. This interesting feature of Asavas-Arishta dosage forms, might be attributed to the residual sugar content generally available in this range. Thus, their disadvantage is also that these beneficial effects can not be delivered to a diabetic patient. This limitation mainly comes from their residual sugar and alcohol contents.

Generally all the Asavas and Arishtas contain 7-12% v/v of alcohol content and about 20% w/w of sugar content. It is well known fact that, in the presence of alcohol the assimilation of carbohydrate such as sugars is very high. This practically means that if a diabetic patient takes any Asavas and Arishtas, the entire sugar content get assimilated into the body and there is a great risk of immediate rise in the blood glucose levels. Considering this risk generally Ayurvedic physicians do not recommend this type of formulation to any diabetic patient. It is equally pertinent that, any herbal drug can not be offered in this advantageous liquid oral dosage forms.

Looking at the process intricacies of Asava/Arishta, inventors presumed that if an anti-diabetic formulation is processed on these lines, it could have the advantages of greater therapeutic potentials coming from Woodfordia fruticosa, spices and self generated alcohol.

With this background the invention seeks to devise a method by which a herbal composition can be prepared without the disadvantages of residual sugar present in the finished product.

Diabetes is a common clinical problem and about 25-30% of population all over the world faces a risk of propensity to this disease. Besides modem medicines, there are many herbal formulations placed in the market with different claims and benefits. Most of the herbal formulations are made available as solid dosage forms like Capsules, Tablets and powders. They are not presented as liquid orals due to risk of their sugar content. This invention allows the manufacturer to prepare herbal composition for diabetes in the form of a liquid oral without disadvantage of sugar content.

### OBJECTS OF THE INVENTION

The main object of the present invention is to provide synergistic oral liquid herbal compositions falling under the category of “asavas” and “arishtas”, useful for management of diabetes, said composition comprising a therapeutically effective amount of plant extracts, self-generated ethanol to the extent of 7 to 12% v/v and having not more than 1 to 3% w/w of sugar content

Another objective of the invention is to prepare herbal compositions using the process pathways of classical Asavas and Arishtas

Yet another object of the invention is to develop a novel process for the manufacture of anti-diabetic compositions ‘Asavas’ and ‘Arishtas’ containing not more than 1 to 3% w/w of residual sugars and 7 to 12% v/v self generated alcohol.

### SUMMARY OF THE INVENTION

The invention provides a synergistic oral liquid herbal composition falling under the category of “asavas” and “arishtas”, useful for management of diabetes, said composition comprising a therapeutically effective amount of plant extracts, self-generated ethanol to the extent of 7 to 12% v/v and having not more than 1 to 3% w/w of sugar content. This invention also provides a novel method for the manufacture of herbal compositions in liquid oral dosage form containing a limited amount of self generated alcohol.

### DETAILED DESCRIPTION OF THE INVENTION

The invention relates to the treatment of an important clinical problem like diabetes wherein herbal formulations have an edge over the conventionally used synthetic drug molecules

In an embodiment, the invention pertains to deliver the specific advantages of Asavas and Arishtas over the conventionally used solid dosage forms. For this purpose the invention seeks to find solution to important limiting factor of the range. The invention goes to the very fundamental aspects of fermentation procedure and identifies simple solution to overcome the issues of residual sugars.

The invention identifies specific drug potentiators to be added to the conventionally used anti-diabetic formulation of herbal origin. Once this is established the invention further worked to formulate the composition using superior process pathways The invention identifies the methods to overcome the issues related to such process pathways, the invention brought in a process, a process ingenuity leading to the development of a new product through a modification in a conventional process.

Thus, the invention provides a synergistic oral liquid herbal composition falling under the category of “Asavas” and “Arishtas”, useful for management of diabetes, said composition comprising a therapeutically effective amount of plant extracts, self-generated ethanol to the extent of 7 to 12% v/v and having not more than 1 to 3% w/w of sugar content.

In an embodiment, the invention provides a synergistic oral liquid herbal composition falling under the category “Asavas” and “Arishtas”, useful for management of diabetes and useful for reducing the blood glucose of mammals, comprising extracts of plants selected from:

a. Momordica charantia (2-5%),

b. Gymenma sylvestre (8-12%),

c. Pterocarpus marsupium (8-12%),

d. Eugenia jambolana (4-10%), and

e. Trigonella foenumgrecum (1-3%).

And having sugar content to the extent of 1 to 3% w/w and self-generated ethanol to the extent of 7 to 12% v/v.

And, optionally comprising extracts/powder of Woodfordia fruticosa (2 to 5%), Piper longum (0.1 to 0.3%), Elettaria cardamomum (0.1 to 0.3%), Myristica fragrans (0.1 to 0.3%) and Ammomum subulatum (0.1 to 0.3%).

In still another embodiment, the invention provides a synergistic oral liquid herbal composition falling under the category “Asavas” and “Arishtas”, useful for management of diabetes, said composition comprising a therapeutically effective amount of plant extracts, ethanol to the extent of 7 to 12% v/v and having not more than 1 to 3% w/w of sugar content and manufactured by the process comprising the steps of:

(a) obtaining the extract of plant parts,

(b) adding nutrients to the extract of step (a) in a manner such that the sugar content in the culture medium does not exceed 20% w/w,

(c) adding micro-organisms capable of fermentation to the culture medium of step (b) and allowing it to ferment until the self-generated ethanol content thereof reaches 7 to 12% v/v and

(d) obtaining the herbal composition having total sugar content of not more than 3% w/w.

In yet another embodiment, the composition falling under the category “Arishtas” and “Asavas”, useful for management of diabetes and useful for reducing the blood glucose of mammals, comprises extracts of:

a. Momordica charantia (2-5%),

b. Gymenma sylvestre (8-12%),

c. Pterocarpus marsupium (8-12%),

d. Eugenial jambolana (4-10%), and

e. Trigonella foenumgrecum (1-3%).

And has sugar content to the extent of 1 to 3% w/w and alcohol to the extent of 7 to 12% v/v;

And, optionally comprises extracts/powder of Woodfordia fruticosa (2 to 5%), Piper longum (0.1 to 0.3%), Elettaria cardamomum (0.1 to 0.3%), Myristica fragrans (0.1 to 0.3%) and Ammomum subulatum (0.1 to 0.3%),

And is manufactured by the method comprising the steps of:

(a) obtaining extract of plant parts,

(b) adding nutrients to the extract of step (a) in a manner such that the sugar content in the culture medium does not exceed 20% w/w,

(c) adding micro-organisms capable of fermentation to the culture medium of step (b) and allowing it to ferment until the self-generated ethanol content thereof reaches 7 to 12% v/v, and

(d) obtaining the herbal composition having total sugar content of not more than 3% w/w.

In an embodiment, the plant parts are obtained by cold infusion or hot decoction methods. The cold infusion method comprises the step of extracting the plant parts in water at a temperature ranging between 20° to 30° C. The hot decoction method comprises the step of extracting the plant parts in water by heating at a temperature in the range of 60 to 90° C.

In still another embodiment, the nutrient in step (b) is a complex nutrient like jaggery or simple sugar like glucose, fructose or any other hexose sugar. This nutrient is in physical forms such as solid or liquid. Further, the nutrients are added by fed batch or batch fermentation method. The nutrients are added in small amounts in regular intervals (at gradient quantities) in fed batch fermentation method. The addition of nutrients in fed batch fermentation method is such that each batch of nutrients added (each gradient) does not impart more than 5% v/v of sugar and the overall quantum of sugar added in the entire process does not exceed 20% w/w. The nutrients are added to the medium in the beginning at once in batch fermentation method. In another embodiment, the microorganisms for fermentation comprise micro-organisms obtained from conventional sources like Woodfordia fruticosa, pure cultures such as baker's yeast, alcohol producing strains of Saccharomyces sp or strains of Saccharomyces cereviceae such as DRF-UDS-004/Wf, DRF-UDS-016/Wf, DRF-UDS-017/Wf or a combination thereof. The strains DRF-UDS-004/Wf, DRF-UDS-016/Wf, DRF-UDS-017/Wf are certain specific strains of Saccharomyces cereviceae. These strains have already been deposited at Microbial Type Culture Collection, Chandigarh, India. These strains are also being deposited at the International depository . . . and bear accession number . . . As such, these strains are accessible and available to the public.

In yet another embodiment, the culture medium is incubated at a temperature ranging between 20 to 37° C. for 2 to 40 days in anaerobic conditions maintaining the pH of the medium from 4 to 6. For best results, the temperature is maintained at 30° C., the incubation is effected preferably for 4 days and the pH of the culture medium is maintained at 4.5.

As such the final sugar content in the herbal composition manufactured according to the process of the invention is not more than 1 to 3% w/w.

The inventive steps for this invention can be divided under three major headings:

1. Identification, development of an orbitarary herbal composition comprising of well known herbal drugs for diabetes. Each 100 ml of the finished product consists of the extract derived from:

| | | |
|---|---|---|
| Gymnema sylvestre (Leaves) | 8-12 | Gm |
| Trigonella foenumgrecum (Seeds) | 1-3 | Gm |
| Prerocarpus marsupium (Heartwood) | 8-12 | Gm |
| Eugenia jambolana (Seeds) | 4-8 | Gm |
| Momordica charantia (Whole fruit) | 2-5 | Gm |

2. The second phase of inventive steps included, identification of certain drug potentiators and identify a process pathway to incorporate the same into a liquid oral dosage form. The following drug potentiators are identified to enhance the antidiabetic activity.

| | | |
|---|---|---|
| Woodfordia fruticosa (Flowers) | 2-5 | Gm |
| Piper longum (Spike) | 0.1-0.3 | Gm |
| Elettaria cardamomum (Fruits) | 0.1-0.3 | Gm |
| Myristica fragrans (Fruit) | 0.1-0.3 | Gm |
| Ammomum subulatum (Fruit) | 0.1-0.3 | Gm |

Once after the selection of active herbal ingredients is affected, there is a need to develop distinct process by which the finished product shall have a lowest amount of residual sugars. During these inventive steps a series of experiments were conducted mainly with reference to inventive step no.3.

These experiments were essentially designed on a simple biological principle that a complex sugar is inverted in to simple sugars by the microorganism to produce alcohol in any medium. When a complex sugar is added in the medium as per traditional formula, only a part of them are inverted and remained as residual sugars in the finished product.

On the other hand, judicious design of the fermentation medium with simple sugars might help to overcome this problem and render the finished product virtually sugar free. Several experiments were conducted using different kinds of sugars such as Jaggery, Sucrose,

Glucose, Invert Syrup etc. These studies invariably proven that the residual sugars can be controlled by means of judicious use of simple sugars in the fermentation medium.

After establishment of new process pathway, further experiments were designed to ascertain the same using different process conditions, variable fermenting organisms and other inter related process parameters.

The present invention provides novel product having hypoglycemic activity comprising of following ingredients.

| Botanical Name | Common Name |
|---|---|
| Gymnema sylvestre | Gurmar |
| Trigonella foenumgrecum | Methi |
| Prerocarpus marsupium | Vijayasar |
| Eugenia jambolana | Jamun seeds |
| Momordica charantia | Karela |
| Woodfordia fruticosa | Dhatki pushpa |
| Piper longum | Pippali |
| Elettaria cardamomum | Elaichi |
| Myristica fragrans | Javitri |
| Ammomum subulatum | Badi Elaichi |

The invention is further illustrated by the following examples which should not be construed as limitations on the inventive scope embodied herein.

### EXAMPLE:1

A combination of Gurmar leaves (Gymnema sylvestre), Methi seeds (Trigonella foenumgrecum), Vijayasar heartwood (Pterocarpus marsupium), Jamun seeds (Eugenia jambolana), Karela (Momordica cherantia) are coarsely ground to small pieces and extracted twice with water using boiling pan. The extract thus obtained was dispensed into 2 sets of Erelynemayor flasks. In one set of flasks jaggery was dissolved as per the traditional text books whereas in another set of flasks invert syrup was added. To both sets of flasks Woodfordia fruticosa and spicy materials such as Piper longum , Elettaria cardamomum, Myristica fragrans, Ammomum subulatum were topped and these flasks were incubated at 30° C. without shaking. The samples were estimated at regular intervals for alcohol generation and residual sugar content.

The results of final round of analysis of this experiment are tabulated below:

| Experiment | Final Sugar Content | Alcohol Content |
|---|---|---|
| Traditional Method | 24% w/w | 10.7% v/v |
| Modified Method | 0.5% w/w | 9.2% v/v |

The amount of alcohol produced at the end of fermentation is 7-11% v/v. Results indicated that, samples prepared as per the textual procedure has shown 20% w/w of residual sugars. However, samples prepared with invert syrup contain less than 1% w/w of residual sugars. The above example clearly indicates that, by an ingenious modification of nutrient in the culture medium, it is possible to control the residual sugar content in the finished product.

### EXAMPLE—2

A combination of Gurmar leaves (Gymnema sylvestre), Methi seeds (Trigonella foenumgrecum), Vijayasar heartwood (Pterocarpus marsupium), Jamun seeds (Eugenia jambolana) and Karela (Momordica charantia) are coarsely ground to small pieces and extracted twice with water using boiling pan. The extract thus prepared is transferred to a Benchtop fermentor BIOFLO 3000. To this extract invert syrup was added. To this medium, powder of Woodfordia fruticosa and spicy materials such as Piper longum, Elettaria cardamomum, Myristica fragrans, Ammomum subulatum were topped over the fermentation medium. The temperature of the fermentation medium was maintained at 30° C. The samples were estimated at regular intervals for alcohol generation and residual sugar content. The results of this experiment are tabulated below:

| Experiment | Final Sugar Content | Alcohol Content |
|---|---|---|
| Modified Method | 0.6% w/w | 9.3% v/v |

The amount of alcohol produced at the end of fermentation is 7-11% v/v and the residual sugars content in the fermentation medium was less than 1% w/w. It is further confirmed that the end results of the process covered by example-1 is same irrespective of the batch size.

### EXAMPLE 3

A combination of Gurmar leaves (Gymnema sylvestre), Methi seeds (Trigonella foenumgrecum), Vijayasar heartwood (Pterocarpus marsupium), Jamun seeds (Eugenia jambolana), Karela (Momordica charantia) and Dhatkipushpa (Woodfordia fruticosa) are coarsely ground to small pieces and extracted twice with water using boiling pan. The extract thus obtained was dispensed into 5 sets of Erelynemayor flasks. Invert syrup was added to all flasks as a source of nutrient. To this spicy materials such as Piper longum, Elettaria cardamomum, Myristica fragrans, Ammomum subulatum were topped and these flasks were inoculated with different microorganisms. The first set of flasks were inoculated with Baker's yeast where as the rest of 4 sets were inoculated with, Saccharomyces cereviceae strains DRF-UDS-004/WF, DRF-UDS-16/WF, DRF-UDS-17/WF individually and a combination of all three strains. As said earlier, these strains have already been deposited at MTCC, Chandigarh, India and are available to he public. All the flasks were incubated under controlled conditions allowing a complex anaerobic fermentation. Samples were estimated at regular intervals for alcohol generation and residual sugar content.

The characteristics of the yeast strain DRF-UDS 004/wf are as under:

a) being sugar resistant,

b) capable of producing alcohol to the extent of about 7 to 11v/v% continuously and consistently in a herbal extraction medium,

c) capable of overcoming resistance conferred by the herbal and spicy ingredients present in the medium,

d) capable of attaining biomass of about 1 to 4 g/l and potentiating and enhancing the therapeutic value of the herbal formulation, and

e) exhibiting the following bio-chemical properties:

i) urease test-positive,

ii) utilization of fructose test-negative,

iii) starch hydrolysis test-negative,

iv) xylose hydrolysis test-negative.

The characteristics of the yeast strain DRF-UDS 016/wf are as under:

a) being sugar resistant,

b) capable of producing alcohol to the extent of about 7 to 11.5 v/v% continuously and consistently in a herbal extraction medium,

c) overcoming resistance conferred by the herbal and spicy ingredients in the medium,

d) capable of attaining biomass of about 1 to 4 g/l and potentiating and enhancing the therapeutic value of the herbal formulation, and

e) exhibiting the following bio-chemical properties:

i) urease test-negative,

ii) utilization of fructose test-positive,

iii) starch hydrolysis test-positive,

iv) xylose hydrolysis test-positive.

The characteristics of the yeast strain DRF-UDS 017/wf are as under:

a) being sugar resistant,

b) capable of producing alcohol to the extent of about 7 to 12 v/v% continuously and consistently in a herbal extraction medium,

c) overcoming resistance conferred by the herbal and spicy ingredients in the medium,

d) capable of attaining biomass of about 1 to 3.5 g/l and potentiating and enhancing the therapeutic value of the herbal formulation, and

e) exhibiting the following biochemical properties:

i) urease test-negative,

ii) utilization of fructose test-positive,

iii) starch hydrolysis test-positive,

iv) xylose hydrolysis test-negative.

The invention also provides a method for the propagation of yeast cultures capable of being used for fermentation of plant extracts, said method comprising the steps of:

a) preparing an aqueous medium containing 10 to 40% sucrose, derived from jaggery,

b) inoculating the medium with a yeast culture,

c) incubating the culture under aerobic conditions at a temperature in the range of 20-37° C. for a period of 24 to 72 hours, and

d) maintaining the culture to obtain a liquid containing 5 to 30 mg of yeast biomass per ml.

The results of this experiment are tabulated below:

| Experiment | Final Sugar Content | Alcohol Content |
|---|---|---|
| Baker's yeast | 0.5% w/w | 9.4% v/v |
| DRF-UDS-004/WF | 0.7% w/w | 9.3% v/v |
| DRF-UDS-16/WF | 0.5% w/w | 9.4% v/v |
| DRF-UDS-17/WF | 0.6% w/w | 9.2% v/v |
| Mixed Culture of DRF- | 0.4% w/w | 9.1% v/v |
| UDS | | |

Experimental results suggested that the process is effective irrespective of type of fermenting microorganism.

### EXAMPLE—4

A combination of Gurmar leaves (Gymnema sylvestre), Methi seeds (Trigonella foenumgrecum), Vijayasar heartwood (Pterocarpus marsupium), Jamun seeds (Eugenia jambolana) Karela (Momordica charantia) and Dhatkipushpa (Woodfordia fruticosa) are coarsely ground to small pieces and extracted twice with water using boiling pan. The extract thus prepared is transferred to a Benchtop fermentor BIOFLO 3000. The nutrient (Invert Syrup) was added in a batch fed mechanism. The medium was inoculated with baker's yeast and spicy materials such as Piper longum, Elettaria cardamomum, Myristica fragrans, Ammomum subulatum were topped over the fermentation medium. The temperature of the fermentation medium was maintained at 30° C. The samples were estimated at regular intervals for alcohol generation and residual sugar content. The amount of alcohol produced at the end of fermentation was 7-11% v/v and the residual sugars content was less than 1% w/w. This particular fed batch mechanism shall have the advantage of avoiding any kind of foaming problem during fermentation. These experimental results further emphasize the effectiveness of the process and the end results of the process are same irrespective of the type of fermentation.

### EXAMPLE:5

To examine the comparative efficacy of two solid oral dosage forms the following extracts were prepared.

Sample-A

The following individual herbs are coarsely ground to small pieces and extracted twice with water using boiling pan. The filtrate is then collected and was dried to obtain a fine powder (Quantities mentioned here are equivalent to 100 ml of liquid oral preparation)

| | | |
|---|---|---|
| Gymnema sylvestre | 8-12 | Gm |
| Trigonella foenumgrecum | 1-3 | GM |
| Prerocarpus marsupium | 8-12 | Gm |
| Eugenia jambolana | 4-8 | Gm |
| Momordica charantia | 2-5 | Gm |

Sample-B

The above extract is divided into two portions and to the one portion the dry powders blend of following herbs was added.

| | | |
|---|---|---|
| Woodfordia fruticosa | 2-5 | Gm |
| Piper longum | 0.1-0.3 | Gm |
| Elettaria cardamomum | 0.1-0.3 | Gm |
| Myristica fragrans | 0.1-0.3 | Gm |
| Ammomum subulatum | 0.1-0.3 | Gm |

Sample C

Sample C was prepared by the novel process mentioned in the description.

Confirmation of Antidiabetic Acivity

Diabetes was induced in 9 rats using Streptozotocin by a single dose administration. These rats were divided into three groups, the first group received the treatment with sample-A, second group treated with sample-B whereas the third group served as an untreated control. Blood sugar levels were estimated in all three groups. Blood sugar levels were controlled in both the test groups. However, the effects were much significant in animals treated with sample-B. This example suggests by addition of powdered Woodfordia fruticosa along with spicy materials enhance the anti-diabetic activity of prime ingredients.

The results are tabulated below:

| Blood glucose levels | Initial | After treatment |
|---|---|---|
| Control group | 183 | 172 |
| Test Group-1 (Sample-A) | 180 | 130 |
| Test Group-2 (Sample-B) | 181 | 110 |

### EXAMPLE-6

To examine the comparative efficacy of solid oral dosage form (Sample-B) and the liquid oral dosage form the following experiment was conducted.

Diabetes was induced in 9 rats using Streptozotocin by a single dose administration. These rats were divided into three groups, the first group received the treatment with sample-B, Second group treated with liquid oral mentioned in example-4, whereas the third group served as an untreated control. Blood sugar levels were estimated in all three groups. Blood sugar levels were controlled in both the test groups. However, the effects were much significant in animals treated with liquid oral. This example suggests that the product produced by the novel process has shown better biological activity than the solid oral dosage form of the same composition. Hence, it is proved that the liquid oral dosage prepared by this process has shown better hypoglycemic activity.

The results are tabulated below:

| Blood glucose levels | Initial | After treatment |
|---|---|---|
| Control group | 182 | 171 |
| Test Group-1 (Sample-B) | 183 | 120 |
| Test group-2 (Sample C) | 180 | 75 |

### EXAMPLE:7

Hypoglycemic effect of the above liquid oral sample was tested in human beings. 10 known diabetic patients were selected and were treated with the sample. Each patient was administered with 10 ml of the liquid twice a day for about 12 weeks. During the treatment regime the patients were asked to take controlled diet. Blood samples were withdrawn for every four weeks and was analysed for blood sugar levels. The results are tabulated as follows.

| | Initial | Blood Sugar | Blood Sugar | Blood Sugar |
|---|---|---|---|---|
| Patients | Blood | Level (After 4 | Level (After 8 | Level (After 12 |
| Number | sugar level | weeks) | weeks) | weeks) |
| Patient 1 | 190 | 170 | 140 | 105 |
| Patient 2 | 185 | 172 | 138 | 110 |
| Patient | 185 | 165 | 142 | 100 |
| Patient 3 | 200 | 175 | 145 | 120 |
| Patient 4 | 192 | 171 | 138 | 103 |
| Patients 5 | 190 | 165 | 130 | 105 |
| Patient 6 | 185 | 155 | 125 | 98 |
| Patient 7 | 180 | 158 | 120 | 95 |
| Patient 8 | 190 | 148 | 126 | 102 |
| Patient 9 | 195 | 150 | 132 | 95 |
| Patient 10 | 180 | 142 | 125 | 98 |

### EXAMPLE.8

The liquid oral preparation was also tested in combination with allopathic oral anti diabetic drug. Each patient was administered with the sample along with conventional oral anti diabetic drugs available in the market. Blood sugar levels were estimated at regular intervals. In all the patients, consistent fall in the blood sugar level was observed. After two months of the treatment the patients were asked to withdraw the conventional oral anti diabetic drug and maintained with the liquid oral drug prepared by the novel process. The results of the study indicate that the drug has a synergistic activity when used in combination with allopathic medicines. This indicates that the drug is effectively reducing the dose of allopathic medicines by maintaining the blood sugar levels within the physiological range.

1. A synergistic oral liquid herbal composition falling under the category of “asavas” and “arishtas”, useful for management of diabetes, said composition comprising a therapeutically effective amount of plant extracts, self-generated ethanol to the extent of 7 to 12% v/v and having not more than 1 to 3% w/w of sugar content.

2. A composition as claimed in claim 1 wherein the plant extracts are selected from
a. Momordica charantia (2-5%),
b. Gymenma sylvestre (8-12%),
c. Pterocarpus marsupium (8-12%),
d. Eugenia jambolana (4-10%), and
e. Trigonella foenumgrecum (1-3%),
and, optionally, comprising extracts/powder of Woodfordia fruticosa (2 to 5%), Piper longum (0.1 to 0.3%), Elettaria cardamomum (0.1 to 0.3%), Myristica fragrans (0.1 to 0.3%) and Ammomum subulatum (0.1 to 0.3%).

3. A synergistic oral liquid herbal composition falling under the category “Asavas” and “Arishtas”, useful for management of diabetes and useful for reducing the blood glucose of mammals, comprising extracts of plants selected from:
a. Momordica charantia (2-5%),
b. Gymenma sylvestre (8-12%),
c. Pterocarpus marsupium (8-12%),
d. Eugenia jambolana (4-10%), and
e. Trigonella foenumgrecum (1-3%),
and having sugar content to the extent of 1 to 3% w/w and self-generated ethanol to the extent of 7 to 12% v/v,
and, optionally, comprising extracts/powder of Woodfordia fruticosa (2 to 5%), Piper longum (0.1 to 0.3%), Elettaria cardamomum (0.1 to 0.3%), Myristica fragrans (0.1 to 0.3%) and Ammomum subulatum (0.1 to 0.3%).

4. A synergistic oral liquid herbal composition falling under the category “Asavas” and “Arishtas”, useful for management of diabetes, said composition comprising a therapeutically effective amount of plant extracts, ethanol to the extent of 7 to 12% v/v and having not more than 1 to 3% w/w of sugar content and manufactured by the process comprising the steps of:
a. obtaining the extract of plant parts,
b. adding nutrients to the extract of step (a) in a manner such that the sugar content in the culture medium does not exceed 20% w/w,
c. adding micro-organisms capable of fermentation to the culture medium of step (b) and allowing it to ferment until the self-generated ethanol content thereof reaches 7 to 12% v/v and
d. obtaining the herbal composition having total sugar content of not more than 3% w/w.

5. A synergistic oral liquid herbal composition as claimed in claim 1, falling under the category “arishtas” and “asavas”, useful for management of diabetes and useful for reducing the blood glucose of mammals, comprising extracts of:
a. Momordica charantia (2-5%),
b. Gymenma sylvestre (8-12%),
c. Pterocarpus marsupium (8-12%),
d. Eugenia jambolana (4-10%), and
e. Trigonella foenumgrecum (1-3%),
and having sugar content to the extent of 1 to 3% w/w and alcohol to the extent of 7 to 12% v/v,
and, optionally, comprising extracts/powder of Woodfordia fruticosa (2 to 5%), Piper longum (0.1 to 0.3%), Elettaria cardamomum (0.1 to 0.3%), Myristica fragrans (0.1 to 0.3%) and Ammomum subulatum (0.1 to 0.3%), said composition being manufactured by a method comprising the steps of:
a. obtaining extract of plant parts,
b. adding nutrients to the extract of step (a) in a manner such that the sugar content in the culture medium does not exceed 20% w/w,
c. adding micro-organisms capable of fermentation to the culture medium of step (b) and allowing it to ferment until the self-generated ethanol content thereof reaches 7 to 12% v/v, and
d. obtaining the herbal composition having total sugar content of not more than 3% w/w.

6. A composition as claimed in claims 3, 4 or 5 wherein the plant parts are obtained by cold infusion or hot decoction methods.

7. A composition as claimed in claim 6 wherein the cold infusion method comprises the step of extracting the plant parts in water at a temperature ranging between 20° to 30° C.

8. A composition as claimed in claim 6 wherein the hot decoction method comprises the step of extracting the plant parts in water by heating at a temperature in the range of 60 to 90° C.

9. A composition as claimed in claim 4 or 5 wherein the nutrient in step (b) is a complex nutrient like jaggery or simple sugar like glucose, fructose or any other hexose sugar.

10. A composition as claimed in claim 9 wherein the nutrient is in physical forms such as solid or liquid.

11. A composition as claimed in claim 9 wherein the nutrients are added by fed batch or batch fermentation method.

12. A composition as claimed in claim 9 wherein the nutrients are added in small amounts in regular intervals (at gradient quantities) in fed batch fermentation method.

13. A composition as claimed in claim 12 wherein the addition of nutrients in fed batch fermentation method is such that each batch of nutrients added (each gradient) does not impart more than 5% v/v of sugar and the overall quantum of sugar added in the entire process does not exceed 20% w/w.

14. A composition as claimed in claim 12 wherein the nutrients are added to the medium in the beginning at once in batch fermentation method.

15. A composition as claimed in claims 3, 4 or 5 wherein the microorganisms for fermentation comprise micro-organisms obtained from conventional sources like Woodfordia fruticosa, pure cultures such as baker's yeast, alcohol producing strains of Saccharomyces sp or strains of Saccharomyces cereviceae such as DRF-UDS-004/Wf, DRF-UDS-016/Wf, DRF-UDS-017/Wf or a combination thereof.

16. A composition as claimed in claim 4 or 5 wherein in step (c) the culture medium is incubated at a temperature ranging between 20 to 37° C. for 2 to 40 days in anaerobic conditions maintaining the pH of the medium from 4 to 6.

17. A composition as claimed in claim 16 wherein the temperature is maintained at 30° C.

18. A composition as claimed in claim 16 wherein the incubation is effected for 4 days.

19. A composition as claimed in claim 16 wherein the pH of the culture medium is maintained at 4.5.

20. A composition as claimed in claims 3, 4 or 5 wherein the final sugar content in the herbal composition is not more than 1% w/w.
